SEQUENCE LISTING (10 sequences)

SEQ ID NO: 1 - protein, 101 aa, Artificial Sequence
  101 Amino acid Tat protein
MEPVDPRLEPWKHPGSQPKTACTNCYCKKCCFHCQVCFMTKALGISYGRKKRRQRRRAHQNSQTHQASLSKQPTSQSRGDPTGPKEQKKKVERETETDPFD

SEQ ID NO: 2 - protein, 86 aa, Artificial Sequence
  86 amino acid Tat mutant
MEPVDPRLEPWKHPGSQPKTACTNCYCKKCCFHCQVCFMTKALGISYGRKKRRQRRRAHQNSQTHQASLSKQPTSQSRGDPTGPKE

SEQ ID NO: 3 - protein, 71 aa, Artificial Sequence
  71 amino acid Tat mutant
MEPVNPSLEPWKHPGSQPKTACTNCYCAKCCFHCQVCFITKALGISYGRAKRRQRRRPPQGSQTHQVSLSK

SEQ ID NO: 4 - DNA, 303 bp, Artificial Sequence
  base sequence of 101 amino acid Tat
atggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcaacccacctcccaatcccgaggggacccgacaggcccgaaggaacagaagaagaaggtggagagagagacagagacagatccattcgat

SEQ ID NO: 5 - DNA, 258 bp, Artificial Sequence
  base sequence of 86 amino acid Tat
atggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcaacccacctcccaatcccgaggggacccgacaggcccgaaggaa

SEQ ID NO: 6 - DNA, 213 bp, Artificial Sequence
  base sequence of 71 amino acid Tat
atggagccagtaaatcctagcctagagccctggaagcatccaggaagtcagcctaaaactgcttgtaccaattgctattgtgcaaagtgttgctttcattgccaagtttgtttcataacaaaagccttaggcatctcctatggcagggcaaagcggagacagcgacgaagacctcctcaaggcagtcagactcatcaagtttctctatcaaag

SEQ ID NO: 7 - DNA, 38 bp, Artificial Sequence
  Primer
gatcggatccaccatggagccagtaaatcctagcctag

SEQ ID NO: 8 - DNA, 31 bp, Artificial Sequence
  Primer
gatcgaattcctttgatagagaaacttgatg

SEQ ID NO: 9 - DNA, 38 bp, Artificial Sequence
  Primer
gatcggatccaccatggagccagtacctagactagagc

SEQ ID NO: 10 - DNA, 35 bp, Artificial Sequence
  Primer
gatcgaattcttccttcgggcctgtcgggtcccct

(19) United States
(12) Patent Application Publication
(10) Pub. No.: US 2007/0073040 A1
(43) Pub. Date: Mar. 29, 2007

(54) Anti-obesity polypeptides

(75) Inventors: Man-Wook Hur, Gyoungki-do (KR); Dong-Kee Lee, Seoul (KR)

Correspondence Address: JONES DAY, 222 EAST 41ST ST, NEW YORK, NY, 10017, US

(73) Assignee: Yonsei University

(21) Appl. No.: 11/518,030

(22) Filed: Sep. 8, 2006

Related U.S. Application Data
(63) Continuation of application No. 10/475,688, filed on Oct. 20, 2003, now abandoned.

(30) Foreign Application Priority Data
Apr. 20, 2001 (KR) 10-2001-0021450

(51) Int. Cl. C07K 14/705; C07H 21/04

(52) U.S. Cl. 530/350; 536/23.5; 435/320.1

(57) ABSTRACT

The present invention relates to anti-obesity polypeptides for restraining the generation of adipose tissue in vivo. The anti-obesity polypeptides can be used as obesity preventive vaccines or treating agents and in curing diabetes.

### BACKGROUND OF THE INVENTION

1. Field of the Invention

Obesity is a pathologic disorder caused by excess fat accumulation in various tissues, particularly in abdominal adipose tissues. Excess food-intake of high fat content, lack of exercise, genetic, environmental and psychological factors are the causes of obesity.

Obesity caused by quality and quantity of feeding food and hereditary, environmental and psychological factors as the society is advanced, is a pathologic disorder resulting from fat accumulation in particularly adipose tissue among tissues. Obesity is regarded as a direct or indirect cause of diabetes mellitus, cardiovascular disease, and short life expectation, and other diseases. Prevention and treatment of obesity is an important health issue for healthy life.

2. Description of the Prior Art

Many studies demonstrated that reduction in fat mass by diet control or regular exercise reduces the above-described health risk dramatically. Unfortunately, diet-control and exercise are often not successful. The failure is strongly associated with increased appetite, preference for highly caloric foods, reduced physical activity, and various genetic factors related with lipid biosynthesis. Especially, it is very hard to treat the obese people with high genetic risk factors. Therefore, a new therapeutic agent that can treat obesity is needed.

Although diet control and exercise may prevent potential obesity patients from being obese, most of them will fall into obese state in the long run. Thus, in order to prevent and treat obesity by a relatively simple therapeutic method, we, in the present invention, invented the relatively simple polypeptides that may be applied to the prevention or treatment of obesity in form of protein drugs or gene therapeutic agents.

For our biochemical and molecular biological studies, we used to prepare polyclonal antibodies by subcutaneous injection of suspension mixture composed of recombinant antigens and adjuvant into the hypoderm of white rabbit about 2-4 times for 4-6 weeks period and by serum collection. In one of such experiments, we found that a specific polypeptide (hereinafter, referred to as “Synleptin”) induces weight loss by more than 20% in rabbits. The main cause of weight loss was due to drastical decrease in fat mass in various abdominal regions or organs, and subcutaneous regions. This polypeptide can be used as an agent preventing or therapeutic agent against obesity.

The present invention provides a biological anti-obesity agent. Anti-obesity hormone, Leptin, protein, diet control, and physical exercise are conventional methods of treating obesity. However, the present invention provides an innovating anti-obesity agent with potent ability to repress the formation of adipose or fat tissue in vivo. More specifically, the present invention relates to the protein that dramatically represses generation of adipose tissue when injected into hypoderm with a protein consisting of 72 or 86 amino acids mixed with adjuvant oil. As partial Tat (trans-activating protein of HIV) polypeptide fragments consisting of 71 or 86 amino acids are showed anti-obesity activities, Tat polypeptides consisting of all or part of 101 amino acids will also have anti-obesity effect. The present invention is based on a novel discovery that Tat proteins repress fat biosynthesis in vivo. AIDS patients may become thin due to Tat protein. Tat polypeptides and its various derivatives can be used as preventive or therapeutic agents against obesity in protein drug or gene therapy agent forms.

### SUMMARY OF THE INVENTION

Accordingly, in order to overcome the above-described problem, the present invention is to provide a biological therapeutic agent for effective prevention or treatment of obesity.

In order to accomplish the above-described objective, the present invention provides a polypeptide shown in SEQ ID NO:1, 2 and 3 or a pharmaceutically acceptable salts thereof.

In the present invention, a polypeptide of SEQ ID NO:1 is a Tat protein consisting of 101 amino acids, a polypeptide of SEQ ID NO:2 is 86 amino acids from Nterminus of Tat, and a polypeptide of SEQ ID NO:3 is 71 amino acids from N-terminus of Tat. However, the polypeptides with anti-obesity activity in various tissues in the present invention include the above-mentioned Tat polypeptides and their diverse mutant type polypeptides (there are various kinds of natural or artificial mutants of Tat), comprising the part or the whole thereof.

The present invention also provides the genes having nucleotide sequences shown in SEQ ID NOS:4-6 that encode the anti-obesity polypeptides, and pcDNA3.0 SynLeptin-1 and -2 or pGEX4T SynLeptin-1 and -2 fusion gene recombinant plasmids.

The polypeptide of the present invention can be used as preventive or therapeutic agents against obesity in various protein drug or gene therapy agent forms.

### BRIEF DESCRIPTION OF THE DRAWINGS

FIG. 1 illustrates weight loss caused by the administration of Synleptin polypeptide with anti-obesity and repression of fat accumulation in various tissues including adipose tissue in rabbits. Compared with the control group, SynLeptin-1 polypeptide induced 20% weight loss by repressing the formation of adipose tissues (see FIG. 3).

FIG. 2 illustrates weigh loss when SynLeptin-1 or 2 polypeptide was injected two times every two weeks into the hypoderm of 12-week-old ZFD rats with non-functional Leptin receptor. After about 30 days, while the control group treated with saline showed 8.5% of weight increase, the group treated with SynLeptin-1 showed 3.5% of weight increase, and, in contrast, the group treated with SynLeptin-2 showed rather 4.3% of weight loss instead.

FIG. 3 shows autopsy results. In FIG. 3A, arrows indicate distribution of adipose tissue in various organs in the test rabbit that SynLeptin-2 polypeptide-Adjuvant mixture was injected three times into the hypoderm every two weeks. In FIG. 3B, arrows indicate distribution of adipose tissue in various organs injected with saline-Adjuvant mixture three times into the hypoderm every two weeks. As shown in pictures of FIG. 3, polypeptides in the present invention markedly reduced fat in various tissues including adipose tissue.

### DETAILED DESCRIPTION OF THE PREFERRED EMBODIMENTS

The present invention will be explained in terms of exemplary embodiments described in detail with reference to the accompanying drawings, which are given only by way of illustration and thus are not limitative of the present invention.

As mentioned above, obesity is a pathologic disorder resulting from excess fat accumulation in various tissues, particularly in adipose tissues. Obesity is a direct or indirect causes of diseases such as diabetes mellitus, cardiovascular disease, and short life-expectation, and other diseases as well. It is reported that the above disease condition of patients can be markedly improved, or even the diseases can be prevented if one can reduced one's weight by 5% of body weight.

The higher vertebrates regulate their size of adipose tissue precisely by articulate hormonal systems. In proportion to increasing body weight due to enlargement of adipose tissue, concentration of the Leptin hormone regulating body weight or adipose tissue mass, is increased in blood, and Leptin binds to the receptor in hypothalamus in the brain. A series of regulator processes that reduces body weight are occurring by the hormone-receptor interaction. The interaction promotes secretion of melanocyte stimulating hormone and also increases number of melanocortin-4 receptor. These processes result in decrease in food-intake by loss of appetite, increased emission of body energy, and stimuli of sympathetic nervous system. On the contrary, in proportion to decreasing body weight due to reduction of adipose tissue, the amount of Leptin hormone are decreased in blood and hypothalamus in the brain senses this condition. A number of regulatory processes that sense the low concentration of Leptin, increase body weight by increasing neuropeptide Y concentration and Y-receptor number. These processes result in increases in food-intake by increasing appetite, decreasing consumption of body energy, decreasing reproductive ability and body temperature, and increasing stimuli of parasympathetic nervous system. Therefore, as mentioned above the precise feedback regulating mechanisms regulate the quantity of fat tissue and the body weight.

Hormone genes playing an essential role in the regulation of body weight include Obese(ob), Diabetes(db), and agouti. Particularly, ob is a 16 kDa polypeptide (called Leptin), which is synthesized in adipose tissue and carried to bloodstream. If mutations such as abnormal expression and function of gene coding the polypeptide or errors on number and function of cell surface hormone receptor are introduced, the size of fat tissue is markedly increased and the body weight is increased.

Previous study has demonstrated that ob polypeptide (Leptin) injected in blood has an obvious effect on obesity treatment in experimental animals. Clinical test for human application is in progress. The clinical result shows that some obese people have lost 7.1 kg of their body weight, and others did not lost weight upon Leptin treatment. The further investigation showed that obesity biology is complex and caused by various genetic defects including ob receptor(db) and ob genes.

An experiment using rats suggests that ob polypeptide may treat type II diabetes mellitus by increasing glucose metabolism, independent of body weight.

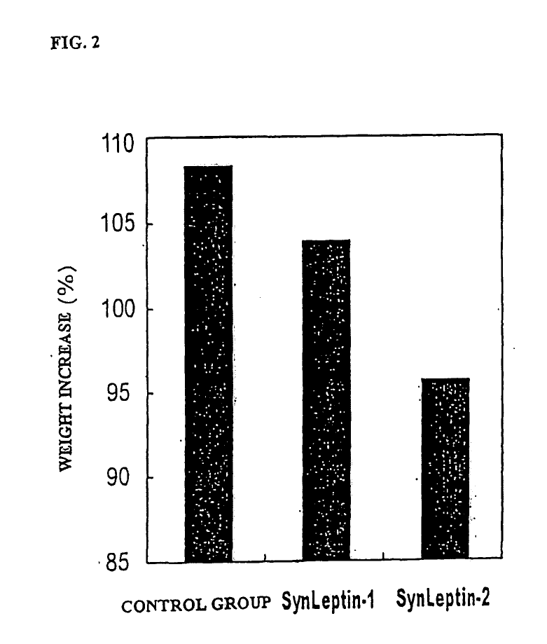

An object of the present invention is to effectively prevent or treat obesity that causes various intractable diseases by repressing accumulation of fat in various tissues. The protein in the present invention likely has therapeutic effect on the obese patients who has resistance to Leptin. Leptin exerts its biological function by binding to its receptor, but because SynLeptin-1 and -2 have cell membrane permeable PTD (protein transduction domain), these polypeptides may repress the generation of fat tissue in spite of changes in the number and function of Leptin receptor. Indeed, the animal experiment with the ZDF rats that have non-functional ob receptor (db) shows loss of body weight upon treatment with Synleptin polypeptides (FIG. 2). Accordingly, the polypeptides in the present invention likely have therapeutic effect on the obese patients who has resistance to Leptin.

Furthermore, while Leptin must be injected daily into blood stream to achieve significant weight loss, SynLeptin-1 and -2 with PTD domain can be administered into our body much easily. For example, two to three times subcutaneous injection of the polypeptide-adjuvant oil mixture is sufficient to maintain thin state more than 150 days.

Although people make various efforts such as diet-control, rigorous exercise, and chemotherapy to prevent and treat obesity, they tend to return to obese state when they stop those efforts. To overcome the above-described problem, Synleptin polypeptides in the present invention is provided to prevent and treat obesity by potently repressing the accumulation or generation of fat in various tissues.

### EXAMPLE 1

### Preparation of pGEX4T-3-SynLeptin-1 and -2 Expression Plasmids and Production of SynLeptin-1 and -2 Polypeptides

PCR reaction on SynLeptin-1 was performed using a sense primer (GATCGGATCCACCATGGAGCCAGTAAATCCTAGCCTAG—SEQ ID NO:7) and an anti-sense primer (GATCGAATTCCTTTGATAGAGAAACTTGATG—SEQ ID NO:8). The PCR condition was as follows. After Tat cDNA was denatured at 94° C. for 5 minutes, 30 cycles of amplification reaction (94° C. 30 sec., 60° C. 30 sec., 72° C. 30 sec.) and final reaction at 72° C. for 5 minutes were preformed. The PCR products were separated from 2.0% agarose gel, purified and then digested with restriction enzymes BamH1 and EcoR1. pGEX4T-3 (Pharmacia Co.) expression vector was digested with the same restriction enzymes (BamH1 andEcoR1) and the digested vector was ligated with Synleptin-1 cDNA/BamH1-EcoR1 fragments using T4 DNA ligase. A ligated mixture was introduced into expression host E. coli BL21(DE3) by transformation.

In order to prepare pGEX4T-3-SynLeptin-2 expression plasmid containing Tat polypeptides of 86 amino acids and produce SynLeptin-2 protein, PCR reaction was performed by using a sense primer (GATCGGATCCACCATGGAGCCAGTACCTAGACTAGAGC—SEQ ID NO:9), an anti-sense primer (GATCGAATTCTTCCTTCGGGCCTGTCGGGTCCCCT—SEQ ID NO:10), and Tat cDNA as template. PCR condition and method preparing pGEX4T-3-SynLeptin-2 were the same with the condition and method described above.

Transformed expression host bacteria (E. coli BL21 DE3) with pGEX4T-3-SynLeptin-1 or-2 were inoculated on TY liquid culture medium containing 100 μg/ml of ampicillin, and cultured overnight. 1 ml of the overnight bacterial culture was added to 100 ml of TY liquid medium containing 100 μg/ml of ampicillin, and then cultured for 1 and ½ hours. The synthesis of fusion protein was induced with 0.2 mM IPTG at 30° C. for 10 hours. SynLetin-1 and -2 proteins were purified by glutathione agarose affinity chromatography.

pcDNA3.0 SynLeptin-1 and -2 mammalian expression vector were prepared by the following process. pcDNA3.0 (Clontech) plasmid was digested with restriction enzyme BamH1 and EcoR1. The BamH1-EcoR1 fragments of Synleptin cDNA-1 or -2 genes (about 220 bp) mentioned above and the digested plasmid were ligated with T4 DNA ligase, and introduced into E. coli DH5α through transformation method. The recombinant plasmids were prepared by an alkaline lysis method.

### EXAMPLE 2

### Animal Experiment on Reducing Body Weight by Repressing the Accumulation of Adipose Tissue

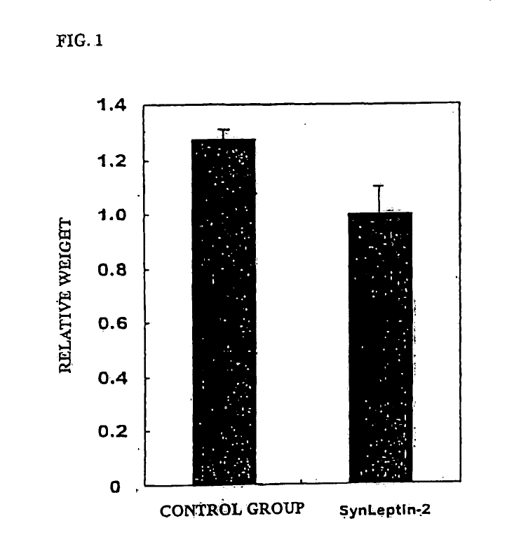

4 white rabbits with 1.2 kg body weight were divided into a control group and an experimental group having two rabbits, respectively. The purified SynLeptin-1 or -2 protein (500-700 μg or saline was mixed with Adjuvant 2 ml (Sigma), and then injected into 4 different parts of the rabbit hypoderm. After 2 weeks, an equivalent amount of the protein-Adjuvant mixture was injected, and then third injection was performed after 2 weeks. Thereafter, body weight loss was measured after 10 days. The result was as follows. The body weight of the experimental group treated with SynLeptin-1 was 1.8 kg and 2.08 kg, respectively (average 1.94 kg), while the body weight of the control group was 2.5 kg and 2.45 kg, respectively (average 2.48 kg). Compared with the control group, the experimental group shows about 22% of body weight loss (see FIG. 1). Also we treated the ZDF rats with nonfunctional Leptin receptor gene with SynLeptin-1 or -2 polypeptides for 30 days (FIG. 2). An experimental group treated with SynLeptin-1 showed 3.5% increase in the body weight. In contrast, the control group showed 8.5% increase of body weight. Especially, an experimental group treated with SynLetin-2 rather showed 4.3% decrease of body weight compared to the control group (FIG. 2).

### EXAMPLE 3

### Autopsy of Experimental Animal

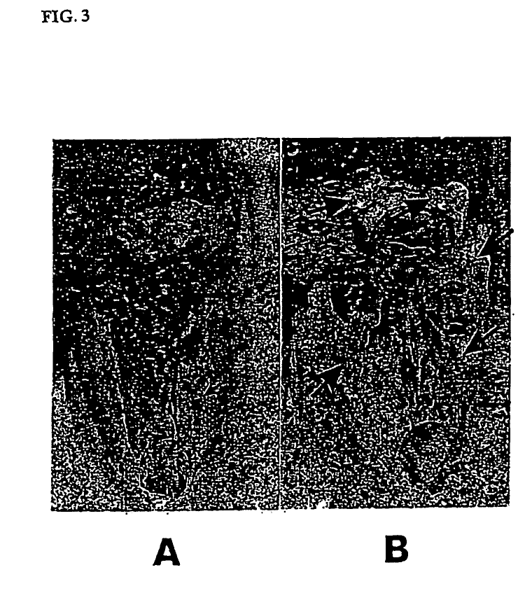

We analyzed the reasons for the decrease in body weight by autopsy and pathological examinations. Rabbits before autopsy showed normal activity or behavior in both control and experimental groups. When the rabbits were sacrificed and autopsy was carried out, no pathological abnormali in their organs was observed. However, remarkable reduction of fat mass not only in abdominal and subcutaneous adipose tissues but also tissues surrounding various organs was evident. Particularly, abdominal adipose tissue was markedly reduced in size (FIG. 3).

The protein in the present invention has a strong effect in preventing and treating obesity by repressing the formation of adipose or fat tissue in various animal tissues. As a result, the protein in the present invention can be used as anti-obesity agents in forms of protein drugs or gene therapy agents.

What is claimed is:

1-6. (canceled)

7. A method for preventing or treating an obesity comprising administering to a subject in need thereof a therapeutically effective amount of a polypeptide having an anti-obesity activity, said polypeptide is selected from the group consisting of:
(a) a polypeptide having the amino acid sequence of SEQ ID NO: 1;
(b) a polypeptide comprising less than all of the amino acid sequence of SEQ ID NO: 1; and
(c) a variant of the polypeptide comprising the amino acid sequence of SEQ ID NO: 1.

8. The method of claim 7, wherein said polypeptide is a polypeptide comprising less than all of the amino acid sequence of SEQ ID NO: 1.

9. The method of claim8, wherein said polypeptide comprises the amino acid sequence of SEQ ID NO: 2 or SEQ ID NO: 3.

10. A method for preventing or treating an obesity comprising administering to a subject in need thereof a therapeutically effective amount of a polynucleotide selected from the group consisting of:
(a) a polynucleotide encoding the sequence of SEQ ID NO: 1;
(b) a polynucleotide encoding the sequence of SEQ ID NO: 2; and
(c) a polynucleotide encoding the sequence of SEQ ID NO: 3.

11. A plasmid vector containing the polynucleotide of claim 10.

12. The plasmid vector of claim 1 1, wherein said plasmid vector is pcDNA3.0 SynLeptin-1, pcDNA3.0 SynLeptin-2, pGEX4T SynLeptin-1 or pGEX4T SynLeptin-2.
